(19) United States
(12) Patent Application Publication
(10) Pub. No.: US 2023/0043103 A1
(43) Pub. Date: Feb. 9, 2023

(54) TEMPORAL CODING OF MARKERS FOR OBJECT TRACKING

(71) Applicant: OXFORD METRICS PLC, Yarnton (GB)

(72) Inventors: Phil Pritchett, Yarnton (GB); Paul Smyth, Yarnton (GB)

(73) Assignee: OXFORD METRICS PLC, Yarnton (GB)

(21) Appl. No.: 17/790,691

(22) PCT Filed: Dec. 18, 2020
(86) PCT No.: PCT/GB2020/053273

§ 371 Date: Jul. 1, 2022

(30) Foreign Application Priority Data
Jan. 6, 2020 (GB) 2000116.0

(51) Int. Cl. G06T 7/70; G06T 7/20

(52) U.S. Cl. CPC G06T 7/70; G06T 7/20; G06T 2207/30204; G06T 2207/10016

(57) ABSTRACT

There is provided a method of motion tracking comprising arranging one or more active marker devices on an object, the active marker devices being configured to emit light and each having an associated temporally repeating pattern comprising a plurality of time frames, controlling the one or more active marker devices to emit light according to their respective temporally repeating patterns, wherein the temporally repeating patterns are such that the active marker device does not emit light during at least one time frame of the plurality of time frames, detecting light emitted by the one or more active marker devices using one or more cameras, and determining a spatial configuration of the object using the light detected by the one or more cameras.

The invention relates to a method of motion tracking using active marker devices. Motion capture and motion tracking systems are used in a wide variety of applications, from film and video game design to medical uses such as gait analysis. These systems typically work by tracking the position of markers attached to various parts of the body of a subject. Markers can be tracked optically by capturing images of the subjects using multiple cameras placed around a motion capture volume, and deducing the three-dimensional positions of the markers within the volume by comparing the video frames recorded by different cameras. Using the positions of the markers, and pre-existing knowledge about the placement of the cameras and of the markers on the subject's body, the motion tracking system can determine the subject's position and posture as a function of time.

Broadly, the markers used can be divided into passive markers and active markers. Passive markers reflect light emitted from light sources around the motion capture volume, appearing bright in the captured images so they can be easily identified. Active markers emit light themselves, enabling them to be identified in the captured images.

A problem with motion tracking systems using optical markers is that markers need to be identified by the tracking system in order to determine which object or part of an object they correspond to, and therefore the position or orientation of that object or part of the object.

One method of distinguishing active markers from one another is to provide clusters of active markers, where each cluster emits light in a different spatial pattern. If these spatial patterns are distinguishable from one another, the clusters can be identified in the captured images. However, motion tracking typically requires a large number of distinguishable markers to track multiple parts of, for example, an actor's body. This is particularly the case where multiple objects (such as actors) are being tracked simultaneously. In such a case, using spatially distinguishable clusters of active markers requires providing many distinct clusters with different spatial patterns of active markers to provide an adequate number of individually identifiable clusters. Producing and providing many different and highly asymmetrical designs of cluster is expensive and inconvenient.

One approach to addressing this problem is to produce marker clusters each having several active markers, and control different clusters to emit light from different subsets of the active markers. This has the advantage that each cluster device is identical, and so easily mass-produced, but individual clusters can still be distinguished from one another by the motion tracking system. However, this approach is still limited in that the number of clusters that can be used simultaneously and still be distinguishable from one another is limited by the number of active markers in each cluster and their arrangement within the cluster. Producing clusters with a large number of active markers in each cluster, such that a large number of distinguishable spatial patterns are available, may also be expensive. It may also lead to the individual markers in the cluster being placed closer together, which makes it harder to distinguish individual markers in images captured at a distance.

It is an object of the invention to provide an improved method of motion tracking. In particular it is an object of the invention to provide a method which can provide a larger number of distinguishable active markers using a smaller number of active markers or designs of marker cluster.

According to a first aspect of the invention, there is provided a method of motion tracking comprising arranging one or more active marker devices on an object, the active marker devices being configured to emit light and each having an associated temporally repeating pattern comprising a plurality of time frames, controlling the one or more active marker devices to emit light according to their respective temporally repeating patterns, wherein the temporally repeating patterns are such that the active marker device does not emit light during at least one time frame of the plurality of time frames, detecting light emitted by the one or more active marker devices using one or more cameras, and determining a spatial configuration of the object using the light detected by the one or more cameras.

By causing the active marker devices to emit light in a temporally repeating pattern, the markers can be identified from the temporal pattern. This means that a larger number of markers can be used simultaneously, and still be uniquely identified from their temporal patterns. This simplifies the design of active marker devices and active marker clusters relative to the case where spatial arrangement of the markers is used for identification. It can also allow the spatial arrangement of the active marker devices to be better optimised for tracking, while still allowing them to be adequately identified.

In an embodiment, the time frames of the temporally repeating patterns all have the same duration.

By having frames with consistent duration, the time to identify a marker is consistent regardless of the point in the pattern at which the marker is first acquired or detected by a camera.

In an embodiment, the temporally repeating patterns are such that the active marker devices emit light during at least one time frame of every pair of consecutive time frames.

By having no consecutive frames where the active marker is not illuminated, consistent tracking of that marker, in two or three dimensions is obtained because the position of the marker can be regularly verified. This minimises the likelihood of losing track of a marker because it is not visible for an extended period of time.

In an embodiment, the plurality of time frames of the temporally repeating patterns of at least a subset of the active marker devices includes tracking frames during which the active marker devices emit light, the tracking frames of the temporally repeating patterns of all of the subset of active marker devices occur simultaneously, and every nth time frame of the temporally repeating patterns of the subset of the active marker devices is a tracking frame, where n is an integer greater than or equal to 2.

By having synchronised tracking frames, all of the active marker devices within the subset are simultaneously visible during the tracking frame. This improves the accuracy of identification of the position and orientation of the object being tracked, because the object is tracked with the largest number of active markers during the tracking frames.

In an embodiment, the active marker devices are controlled such that for each of the plurality of time frames of the temporally repeating patterns, the active marker devices either emit light for the entire duration of the time frame, or do not emit light for the entire duration of the time frame.

By having the active markers illuminated for the entire frame, the available light during the frame is maximised, improving the ability of the cameras of the motion tracking system to receive sufficient light to detect the active marker in each video image.

In an embodiment, the temporally repeating patterns are such that the active marker devices emit light for at least 25% of a duration of the temporally repeating patterns. Having active marker devices illuminated for a minimum proportion of the time improves accuracy and consistency of tracking by reducing the likelihood of losing track of the active marker during a long period in which it does not emit light.

In an embodiment, the step of controlling the active marker devices comprises controlling the active marker devices such that the time frames of the temporally repeating patterns are synchronised with the capturing of images by the one or more cameras.

Synchronising the time frames of the temporally repeating patterns with the frames of the camera makes it easier to determine whether a marker is illuminated or not during any given frame, thereby making it easier to accurately identify any given marker device from its temporally repeating pattern.

In an embodiment, the active marker devices comprise a receiver configured to receive wireless signals, and controlling the active marker devices comprises transmitting a synchronisation signal to the active marker devices and controlling the active marker devices to emit light based on the synchronisation signal.

Having a wirelessly transmitted synchronisation signal means that the cameras and marker devices will remain consistently synchronised even during long motion capture sessions.

In an embodiment, arranging one or more active marker devices on the object comprises arranging a plurality of active marker devices on the object having an associated plurality of temporally repeating patterns, and the temporally repeating pattern of a first active marker device is different from the temporally repeating pattern of at least one other active marker device.

Providing multiple active marker devices with different patterns provides several distinguishable tracked points on an object. This can be used to determine more information about the spatial arrangement of the object. For example, a pose of a person can be determined if multiple markers are provided on different limbs, or an orientation of a rigid object can be determined.

In an embodiment, the temporally repeating patterns of the plurality of active marker devices are such that, at any point in time, at least 30% of the active marker devices emit light.

Ensuring that at least 30% of the marker devices are illuminated at all times reduces the likelihood of losing track of the object, by ensuring that a minimum number of tracking points are always available.

In an embodiment, all of the temporally repeating patterns in the plurality of temporally repeating patterns have the same duration.

Having the same duration of pattern for all of the active marker devices means that the time to identify a marker is consistent between different active markers, simplifying the identification and tracking of multiple active markers.

In an embodiment, each of the active marker devices within a marker cluster has a fixed spatial relationship with the other active marker devices within the same cluster.

Using marker clusters allows for additional functionality, such as determining the orientation of the cluster, thereby improving the ability to determine the spatial arrangement of the object.

In an embodiment, all of the active marker devices within a marker cluster are mounted on a single rigid surface.

Using marker clusters with a fixed spatial relationship between active marker devices simplifies the process of arranging clusters on an object in a particular spatial arrangement, and makes determination of, for example, the orientation of the cluster more reliable.

In an embodiment, arranging one or more marker clusters on the object comprises arranging a plurality of marker clusters on the object, and the plurality of temporally repeating patterns of active marker devices in a first cluster is different from the plurality of temporally repeating patterns of active marker devices in at least one other cluster.

Having several different clusters provides for distinguishable points for tracking the object or parts of the object in order to determine its spatial configuration. Distinguishing between the clusters using the plurality of temporally repeating patterns means that a greater number of identical clusters can be distinguished using different combinations of the available temporally repeating patterns. This provides greater flexibility and allows more parts of an object to be tracked simultaneously.

In an embodiment, for at least one of the one or more marker clusters, the temporally repeating patterns of all the active marker devices in the marker cluster are the same.

Using the same temporally repeating pattern for all of the active marker devices within the same cluster means that the cluster can be identified from any of the active markers within the cluster, improving the robustness of identification.

In an embodiment, arranging one or more marker clusters on the object comprises arranging a plurality of marker clusters on the object, the plurality of temporally repeating patterns of first active marker devices in a first marker cluster is the same as the plurality of temporally repeating patterns of second active marker devices in a second marker cluster, and a spatial arrangement on the object of the first active marker devices is different from a spatial arrangement on the object of the second active marker devices. By distinguishing between marker clusters using spatial arrangements where their plurality of temporally repeating patterns is the same, the number of clusters that can be simultaneously tracked is increased.

In an embodiment, arranging one or more active marker devices on the object comprises arranging one or more marker clusters on the object, the marker clusters comprising at least one active marker device having an associated temporally repeating pattern, and at least one fixed marker device configured to emit light, and the method further comprises controlling the fixed marker device to continuously emit light.

By using fixed marker devices within a cluster, the likelihood of losing track of the cluster is reduced. The cluster can be identified from the temporally repeating pattern of the active marker devices, and located using the fixed marker devices even at a point in the temporally repeating pattern where the active marker device does not emit light.

In an embodiment, determining a spatial configuration of the object further comprises determining an identity of at least one of the marker clusters based on the pluralities of temporally repeating patterns of the one or more marker clusters. Distinguishing clusters using the plurality of temporally repeating patterns of the cluster can increase the number of simultaneously distinguishable clusters by using different combinations of the available temporally repeating patterns for different clusters.

In an embodiment, determining a spatial configuration of the object further comprises using information about the spatial relationship of active marker devices relative to other active marker devices within the same cluster.

Using information about the spatial arrangement of markers within the cluster can give additional information about the orientation of the cluster, and therefore the orientation of the object or part of the object on which the cluster is arranged.

In an embodiment, determining a spatial configuration of the object comprises determining an identity of at least one of the active marker devices based on the temporally repeating patterns of the active marker devices.

Identifying the active markers based on their temporally repeating patterns increases the number of simultaneously distinguishable clusters, allowing a greater number of objects or parts of an object to be tracked simultaneously.

In an embodiment, determining a spatial configuration of the object comprises determining a position and/or orientation of one or more active marker devices.

Determining a position and/or orientation of the active marker devices allows the position and/or orientation of the object (or part of an object) on which the active marker device is arranged to be deduced.

In an embodiment, determining a spatial configuration of the object further comprises using information about the arrangement of the active marker devices on the object.

Using information about the arrangement of the active marker devices on the object allows the spatial configuration of the object to be determined more accurately, because the configuration of the object relative to the active marker device can be deduced.

In an embodiment, arranging one or more active marker devices on the object comprises arranging a plurality of active marker devices on the object, and using information about the arrangement of the active marker devices on the object comprises using constraints on the relative positions and/or orientations of the active marker devices, the constraints being based on the arrangement of the active marker devices on the object.

Such constraints can be used to improve accuracy of the determined spatial configuration of the object, because the identities and/or the positions and/or orientations of the markers can be corrected based on the constraints. For example, if the identity of an active marker device is uncertain, its identity could be narrowed down by determining how close it is to another active marker device of known identity, and determining if that distance is consistent with constraints on the relative positions of the active marker devices.

There is further provided a motion tracking system implementing the first aspect of the invention. The motion tracking system comprises one or more active marker devices configured to be arranged on an object, the active marker devices being further configured to emit light and each having an associated temporally repeating pattern comprising a plurality of time frames, a control system configured to control the active marker devices to emit light according to their respective temporally repeating patterns, wherein the temporally repeating patterns are such that the active marker devices do not emit light during at least one time frame of the plurality of time frames, one or more cameras configured to detect light emitted by the active marker devices, and determining means configured to determine a spatial configuration of the object using the light detected by the one or more cameras.

There is further provided an active marker device for use in the motion tracking system. The active marker device comprises a light emitting unit configured to emit light, receiving means configured to receive wireless signals from the control system of the motion tracking system, and control means configured to control the emission of light by the light emitting unit based on the wireless signals received by the receiving means. According to a second aspect of the invention, there is provided a method of motion tracking comprising arranging a plurality of active marker devices on an object, the active marker devices being configured to emit light, detecting light emitted by the plurality of active marker devices using one or more cameras, and determining a spatial configuration of the object using the light detected by the one or more cameras, wherein determining a spatial configuration of the object comprises using information about the arrangement of the active marker devices on the object to define constraints on the relative positions of the active marker devices, and identifying at least a subset of the plurality of active marker devices using the light detected by the one or more cameras and the constraints on the relative positions of the active markers.

Using constraints on relative positions of active markers can be used to improve accuracy of the determined spatial configuration of the object, because the identities and/or the positions and/or orientations of the markers can be corrected based on the constraints. For example, if the identity of an active marker device is uncertain, its identity could be narrowed down by determining how close it is to another active marker device of known identity, and determining if that distance is consistent with constraints on the relative positions of the active marker devices.

In an embodiment, the method further comprises determining a position and/or orientation of the plurality of active marker devices, and arranging a plurality of active marker devices on an object comprises arranging a first plurality of active marker devices on a first object and arranging a second plurality of active marker devices on a second object, and identifying at least a subset of the plurality of active marker devices comprises determining whether an active marker device is part of the first plurality of active marker devices or part of the second plurality of active marker devices using the position and/or orientation of the active marker device and the constraints on the relative positions of the active marker devices.

The constraints on relative positions of the active marker devices can be used to distinguish between markers on first and second objects. This further increases the number of points that can be simultaneously tracked by allowing markers that are otherwise indistinguishable to be reused on different objects by identifying the active marker devices using their relationship to other active marker devices.

In an embodiment, the first plurality of active marker devices comprises a unique subset of one or more active marker devices, the unique subset being distinguishable from any other subset of the active marker devices.

Providing a unique subset in each of the first and second pluralities provides a convenient and robust way to distinguish the pluralities of active marker devices, and the other active marker devices within the pluralities can be identified using the constraints by their relationship to the unique subset.

In an embodiment, the unique subset comprises a plurality of active marker devices, and the active marker devices within the unique subset are arranged in a unique spatial configuration, the unique spatial configuration being distinguishable from the spatial configuration of any other subset of the active marker devices.

A unique spatial arrangement is a convenient way to distinguish the unique subset in the images captured by the cameras, and allows the unique subset to be identified rapidly, for example from a single image.

Embodiments of the invention will now be described, by way of example only, with reference to the accompanying drawings in which corresponding reference symbols represent corresponding parts, and in which:

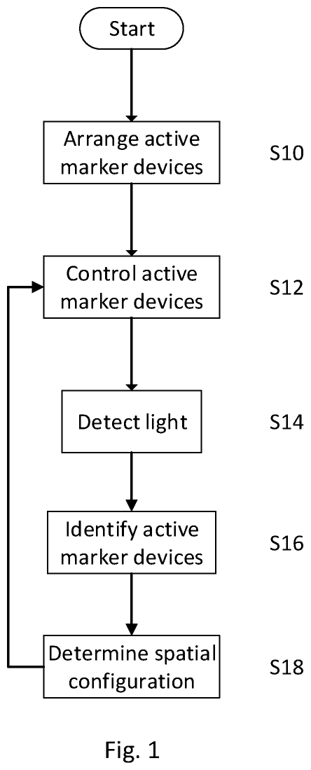

FIG. 1 is a flowchart of a method of motion tracking according to the first aspect of the invention;

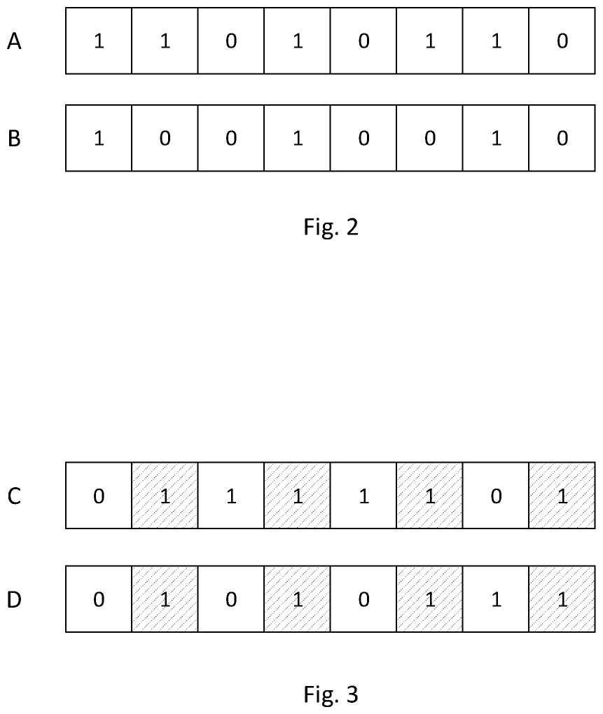

FIG. 2 is an illustration of two temporally repeating patterns;

FIG. 3 is an illustration of temporally repeating patterns using tracking frames;

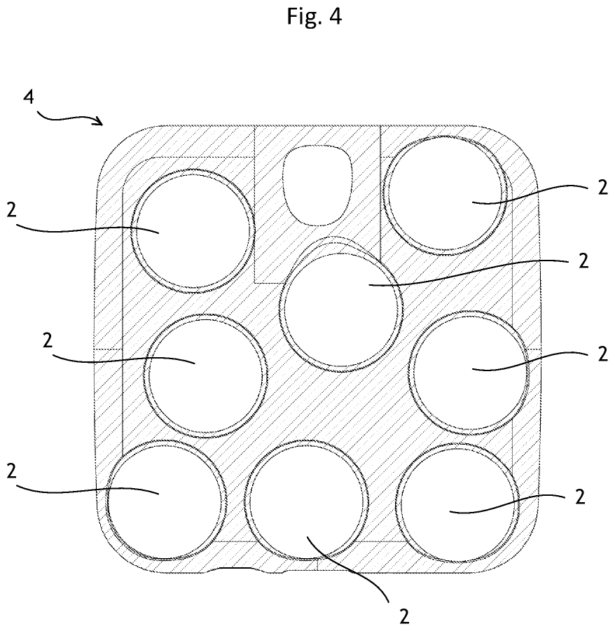

FIG. 4 shows a marker cluster device;

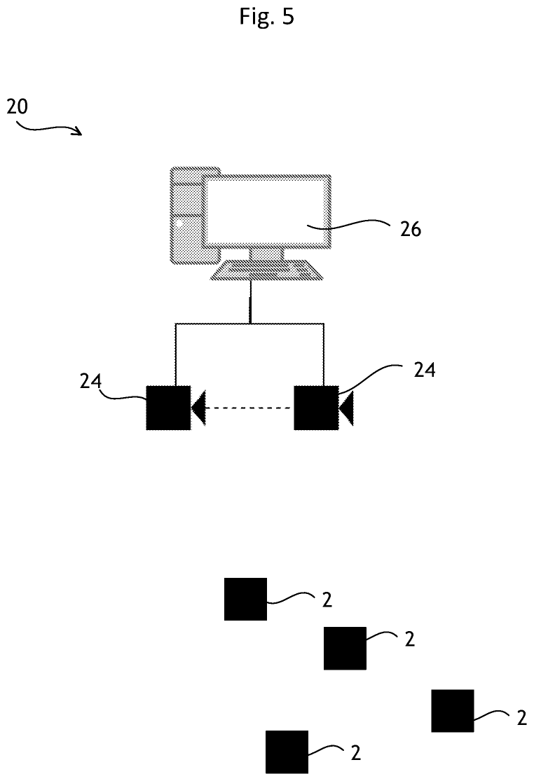

FIG. 5 is a schematic of a motion tracking system.

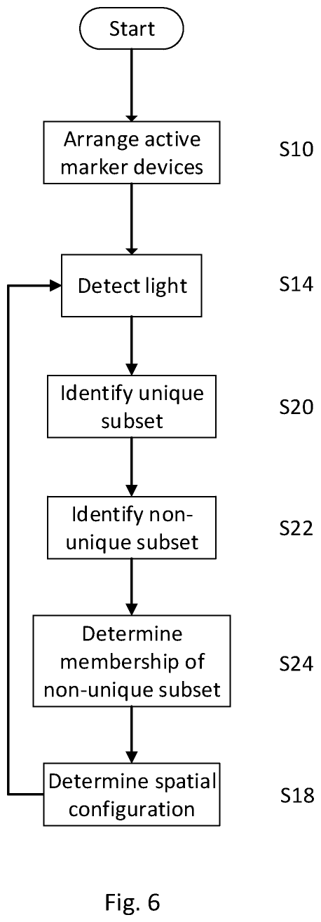

FIG. 6 is a flowchart of a method of motion tracking according to a second aspect of the invention.

A method of motion tracking is provided, as shown in FIG. 1. The motion tracking may be real-time tracking, where the spatial configuration of the object is determined and reported continuously during a motion capture session. Alternatively, the motion tracking may be offline motion tracking, where the spatial configuration of the object is determined from a recording of a motion capture session after the session has concluded. The method involves flashing active markers, such as LEDs, with a temporal identification pattern (coding) such that they can be identified, and this identification is then used to initialise the tracking of an object. With the active marker coding proposed, benefits can be realised in terms of the design of active marker devices. For example, active marker devices can be physically identical, with benefits for manufacturing, stocking and management. Further, when attaching active marker devices to articulated objects, requirements to place the active marker devices in asymmetrical arrangements can be relaxed, improving ease-of-use, and management.

At step S10, the method comprises arranging one or more active marker devices on an object. The active marker devices are attached to a particular position on the object, for example using adjustable straps, adhesive, or other means of attachment. These active marker devices are then used as a source of observations for the tracking of rigid or articulated objects on which active marker devices have been placed. Examples of objects that may be tracked include a human, an animal, or non-living objects such as a robot or vehicle. Where a human is tracked, the active marker devices may be attached to clothing worn by the human. In an embodiment, the method is also used to determine the position of an immobile object, for example as a reference point against which to compare the tracking of a mobile object.

The active marker devices are configured to emit light, and at step S12 the method comprises controlling the one or more active marker devices to emit light as discussed further below. In an embodiment, the active marker devices comprise a light emitter (or light emitting unit), such as a light emitting diode, configured to emit light. Light emitting diodes are an advantageous choice of light emitter due to their compact size and low power consumption. In an embodiment, the light emitters comprise optics configured to increase the spread of light emitted by the light emitter, such that the active marker devices are visible from a greater range of angles. In an embodiment, the active marker devices comprise a battery, such that they can be operated without requiring an external wired power connection. In an embodiment, the active marker devices comprise a receiver (or receiving means) configured to receive wireless signals, for example radio-frequency, microwave-frequency, or infra-red signals. Where the method is carried out using a motion tracking system, the wireless signals are transmitted from the control system of the motion tracking system, and the active marker device further comprises control means configured to control the emission of light by the light emitting unit based on the wireless signals received by the receiving means. Using the wireless signals in this way allows the active marker devices to be controlled remotely during a motion capture session, improving setup time and reducing time to reconfigure the active marker devices if required. It can also allow the active marker devices to receive synchronisation signals, as discussed further below.

The method further comprises a step S14 of detecting light emitted by the one or more active marker devices using one or more cameras. In an embodiment, the cameras are placed at known positions around a motion capture volume in which the object being tracked can move. In an embodiment, at least two cameras are used, optionally at least three cameras, optionally at least four cameras. In an embodiment, the cameras are calibrated cameras, where properties of the camera, its image sensor, and its optics are known. Examples of properties that may be known for a calibrated camera include parameters relating to lens distortion, location of the camera in the scene, focal length of the lens, and so on. These parameters can be used, for example, to correct distortion of captured images or as part of estimating the size and distance of objects in an image.

Each active marker device has an associated temporally repeating pattern. In an embodiment, the temporally repeating pattern has a duration of at least 5 milliseconds, optionally at least 50 milliseconds, optionally at least 100 milliseconds, optionally at least 250 milliseconds, optionally at least 500 milliseconds, optionally at least 1 second. The temporally repeating pattern comprises information on when the active marker device should emit light and when it should not emit light for each point in time during the temporally repeating pattern. It can be considered as a series of instructions on how to emit light from the active marker devices over the duration of the temporally repeating pattern. In an embodiment, the temporally repeating pattern is stored in a memory in the active marker device. In an embodiment where the active marker device comprises a wireless receiver, the temporally repeating pattern may be transmitted to the active marker device by an external controller.

The method comprises a step S20 of controlling the one or more active marker devices to emit light according to their respective temporally repeating patterns. When the duration of the temporally repeating pattern has passed, so that the active marker device has emitted light according to each point in the duration of the temporally repeating pattern, control will return to the beginning of the temporally repeating pattern. The temporally repeating patterns loop over a time window longer than the duration of the temporally repeating pattern, so that the behaviour of the active marker device over a period of time longer than the duration of the temporally repeating pattern will comprise a series of repetitions of the behaviour over the duration of the temporally repeating pattern.

The temporally repeating pattern comprises a plurality of time frames. In an embodiment, the temporally repeating pattern comprises at least 2 time frames, optionally at least 5 time frames, optionally at least 10 time frames, optionally at least 20 time frames, optionally at least 50 time frames, optionally at least 100 time frames. In an embodiment, each time frame immediately follows the preceding time frame, so there is no time between time frames which is not assigned to either the preceding or following time frame. Alternatively, a temporal gap may be provided between consecutive time frames. Each time frame has a duration. In an embodiment, the duration of each time frames is at least 1 millisecond, optionally at least 5 milliseconds, optionally at least 10 milliseconds, optionally at least 20 milliseconds, optionally at least 30 milliseconds, optionally at least 50 milliseconds. In an embodiment, the time frames of the temporally repeating patterns all have the same duration. Each time frame comprises information on the intensity of light to be emitted by the active marker devices during the time frame. In an embodiment, the intensity of light emitted by the active marker devices during each frame is either maximum intensity, or minimum (optionally zero) intensity. In such an embodiment, the intensity of light can be expressed as either on or off, and notated as 1 or 0. Examples of temporally repeating patterns notated in this manner are shown in FIGS. 2 and 3. In an embodiment, the active marker devices either emit light for the entire duration of the time frame, or do not emit light for the entire duration of the time frame. The temporally repeating patterns are such that the active marker device does not emit light during at least one time frame of the plurality of time frames. This allows the method to recognise that the light emitted by the active marker device is varying in time.

The design of the temporally repeating patterns is determined based on a number of factors. The design is a trade-off between the percentage illumination (that is, the percentage of the duration of the temporally repeating pattern for which the active markers emit light), and the number of identities supported by the available patterns. The number of distinguishable patterns available can be increased by increasing the length (duration) of the temporally repeating pattern. However, increasing pattern length may increase the time it takes to identify an active marker, and the risk that the marker becomes occluded while it is unidentified. The number of distinguishable patterns can also be increased by allowing a wider range of percentage illuminations, i.e. permitting patterns in which the active marker device emits light for a smaller percentage of the duration of the temporally repeating pattern. However, it is desirable to choose patterns in which the active marker devices are illuminated for a very high fraction of the time, as active markers can only be detected by the cameras when they emit light, and the method relies on detecting their position for object tracking as well as identifying the active markers. In an embodiment, the temporally repeating patterns are such that the active marker devices emit light for at least 25% of a duration of the temporally repeating patterns, optionally at least 50%, optionally at least 60%, optionally at least 70%, optionally at least 80%. Allowing patterns with low percentage illumination increases the number of available patterns, but may make it harder to accurately position the markers in the motion capture volume, and reduce the accuracy and reliability of tracking of the object. Further, to preserve tracking continuity, the temporally repeating patterns should preferably be allowed to only be zero for a very short time, e.g. at most three time frames, optionally at most two time frames, optionally only a single time frame. In an embodiment, the temporally repeating patterns are such that the active marker devices emit light during at least one time frame of every pair of consecutive time frames. For example, the temporally repeating pattern labelled A in FIG. 2 conforms to this requirement, whereas the temporally repeating pattern B does not.

Therefore, the design of the temporally repeating patterns must balance the need to provide an adequate number of distinguishable patterns, and the need to provide a sufficiently high percentage illumination that tracking accuracy and reliability is not impacted. Different strategies can be chosen to minimise the effects on tracking of temporally coded active marker devices being switched off, depending on considerations including the shape and topology of the item, placement of the active marker devices on that item, the object being tracked, and the tracking quality required. These factors may have a bearing on the optimal temporally repeating patterns for each of the active marker devices placed on the object. They may affect the design of patterns globally (i.e. creating a constraint for all of the active marker devices being used), or individually (i.e. affecting the design of the temporally repeating pattern for a particular active marker device but not all of those simultaneously in use).

In an embodiment, the plurality of time frames of the temporally repeating patterns of at least a subset of the active marker devices includes tracking frames during which the active marker devices emit light, and the tracking frames of the temporally repeating patterns of all of the subset of active marker devices occur simultaneously. In an embodiment where the active marker device comprise a wireless receiver, the active marker devices may be synchronised using a synchronisation signal transmitted to the active marker devices. This means that the object is tracked with the maximum number of active marker devices during the tracking frames, thereby improving the accuracy and reliability of the tracking. In this embodiment, every nth time frame of the temporally repeating patterns of the subset of the active marker devices is a tracking frame, where n is an integer greater than or equal to 2. In other words, each tracking frame is separated from the next tracking frame by n−1 intervening time frames. This effectively gives the best possible tracking at a lower frame rate, because the tracking frames can be used to track the object normally (i.e. as if the active marker devices were continuously emitting light) at a fraction 1/n of the frame rate of the temporally repeating pattern, while the intervening frames are used to identify the active marker devices. A very conservative approach is to choose n=2, ensuring that every active marker device in the system is illuminated on even frames, and only odd frames are allowed to be switched off (or vice versa). An example of this implementation is shown in FIG. 3. Temporally repeating patterns C and D both have tracking frames every other frame, shown as the hatched frames. The patterns C and D are nonetheless distinguishable due to the differences in the intervening frames.

In an embodiment, the step of controlling the active marker devices comprises controlling the active marker devices such that the time frames of the temporally repeating patterns are synchronised with the capturing of images by the one or more cameras.

Synchronising the cameras and active marker devices reduces the chance of errors in identifying a marker device. For example, if a time frame of the temporally repeating pattern overlaps two consecutive images, it may appear that the active marker device is emitting light in both the first or second image at reduced intensity. In this situation, the camera may be unable to distinguish an active marker device emitting light in alternate time frames from an active marker device emitting light continuously at reduced intensity.

In such an embodiment, the frame rate at which the cameras capture images is the same as the frame rate of the temporally repeating pattern, and the start of each time frame occurs simultaneously with the capturing of an image by the cameras (i.e. with the opening of the camera shutter). In an embodiment, the time frames of the temporally repeating patterns have a duration equal to the time between the capturing of consecutive images by the one or more cameras. Therefore, there are no gaps in time between consecutive time frames, and the maximum amount of light is available to the cameras during time frames in which the active marker device emit light. This additionally minimises the capture of unwanted background light that would occur from cameras receiving light during such a gap between time frames. In an embodiment, the time frames of the active marker devices and the capturing of images by the cameras are synchronised using internal clocks of the active marker devices and cameras. In an embodiment, controlling the active marker devices comprises transmitting a synchronisation signal to the active marker devices and controlling the active marker devices to emit light based on the synchronisation signal. In such an embodiment, the active marker devices comprise wireless receivers, and the cameras and active marker devices are synchronised using the synchronisation signal transmitted to both the cameras and active marker devices. In an embodiment, the synchronisation signal comprises a global frame number, and each time frame of the temporally repeating patterns of each active marker device is associated with a global frame number. In an embodiment, the illumination of an active marker device (i.e. whether or not it emits light in that time frame) can be determined from the global frame number. This can aid in identification of the active marker devices, if it can be determined from the global frame number whether or not a given active marker device should be illuminated and emitting light.

In an embodiment, arranging one or more active marker devices on the object comprises arranging a plurality of active marker devices on the object having an associated plurality of temporally repeating patterns, and the temporally repeating pattern of a first active marker device is different from the temporally repeating pattern of at least one other active marker device. Therefore, the first and second active marker devices can be distinguished from one another by their temporally repeating patterns. In an embodiment, all of the temporally repeating patterns in the plurality of temporally repeating patterns have the same duration. This can improve the accuracy and reliability of tracking, by allowing other constraints on the temporally repeating patterns to be more easily implemented, for example the tracking frames discussed above. In an embodiment, when treated as independent, temporally repeating patterns of the plurality of active marker devices are designed so as to be minimally correlated in terms of simultaneous invisibility, i.e. all of the active marker devices should never be simultaneously disabled. In an embodiment, the temporally repeating patterns of the plurality of active marker devices are such that, at any point in time, at least 30% of the active marker devices emit light, optionally at least 50%, optionally at least 60%, optionally at least 70%, optionally at least 80%. This ensures a minimum number of active marker devices are illuminated in every image captured by the cameras, so the object can always be tracked accurately and reliably.

Even when active markers are identified using temporal coding, active marker devices may be arranged in clusters on the object, as discussed above in relation to spatial identification of markers. In an embodiment, arranging one or more active marker devices on the object comprises arranging one or more marker clusters on the object, the marker clusters comprising a plurality of active marker devices having an associated plurality of temporally repeating patterns. Active marker devices within a marker cluster are associated with one another for the purposes of identifying and tracking the active marker devices. In an embodiment, the active marker devices within a cluster are physically associated with one another, such that each of the active marker devices within a marker cluster has a fixed spatial relationship with the other active marker devices within the same cluster. This could be achieved by physically connecting multiple active marker devices together, or providing a single marker cluster device comprising several active marker devices that separately emit light. An example of a marker cluster device 4 is shown in FIG. 4. The marker device 4 of FIG. 4 comprises eight active marker devices 2 provided on the marker cluster device 4 in a spatially asymmetric arrangement. In an embodiment, all of the active marker devices within a marker cluster are mounted on a single rigid surface. Active markers within the same cluster are physically proximate so they can be easily identified as belonging to the same single marker cluster in the images captured by the cameras. In an embodiment, the maximum distance between any two active markers within the same marker cluster is at most 50 centimetres, optionally at most 20 centimetres, optionally at most 10 centimetres, optionally at most 5 centimetres, optionally at most 2 centimetres.

When marker clusters are used, it is only necessary to identify the marker cluster as a whole, and not each individual active marker device within the marker cluster. This can provide advantages in terms of robustly and accurately determining the positions of markers when temporal coding is used. In an embodiment, for at least one of the one or more marker clusters, the temporally repeating patterns of all the active marker devices in the marker cluster are the same. This can improve the accuracy and robustness of identification of a marker cluster, because the cluster can be identified from any one of the active marker devices within the cluster, even if one or more of the active marker devices in the cluster are occluded.

In an embodiment, arranging one or more marker clusters on the object comprises arranging a plurality of marker clusters on the object, and the plurality of temporally repeating patterns of active marker devices in a first cluster is different from the plurality of temporally repeating patterns of active marker devices in at least one other cluster. Because it is only necessary to identify the marker cluster, and not all of the individual active marker devices, marker clusters can be distinguished even if individual active marker devices within a first marker cluster have the same temporally repeating patterns as individual active marker devices in other marker clusters. This can further increase the number of points that can be tracked simultaneously, because marker clusters can be distinguished by having different combinations of the available temporally repeating patterns.

Similarly as discussed above, it is advantageous for reliable tracking of a marker cluster to minimise simultaneous invisibility of the active marker devices within the marker cluster. Reliable tracking of a marker cluster can be achieved if at least three active marker devices within the cluster are illuminated at any given time. In an embodiment, this can be achieved by choosing the temporally repeating patterns of the active marker devices within a cluster to ensure that at least three active marker devices, optionally at least four, optionally at least five active marker devices are simultaneously illuminated at all times. Alternatively or additionally, multiple permanently-illuminated active marker devices can be deployed on the object to guarantee the object's trackability. In such an embodiment, the marker clusters comprise at least one active marker device having an associated temporally repeating pattern, and at least one fixed marker device configured to emit light, and the method further comprises controlling the fixed marker device to continuously emit light. These fixed marker devices would not be identifiable in isolation, but in conjunction with knowledge of the object's geometry, and with one or more active marker devices on the object being temporally coded, the identities of all the active marker devices on the object can be determined. The marker cluster as a whole can be identified using the temporally repeating pattern of the active marker device, but the marker cluster remains visible in every image captured by the cameras, improving accuracy of determination of the marker cluster position and/or orientation.

As discussed above, geometrical properties (generally based on asymmetry) are one source of marker identification. Temporal coding patterns provide another source of marker identification. If, for example, a marker cluster is asymmetrical in the spatial arrangement of active marker devices within the cluster, and there are multiple instances of marker clusters with the same spatial arrangement being tracked, then the marker clusters can be distinguished using the temporally repeating patterns. Therefore, in an embodiment, arranging one or more marker clusters on the object comprises arranging a plurality of marker clusters on the object, a relative spatial arrangement on the object of first active marker devices in a first marker cluster is the same as a relative spatial arrangement on the object of second active marker devices in a second marker cluster, and the plurality of temporally repeating patterns of the first active marker devices is different from the plurality of temporally repeating patterns of the second active marker devices. This will make tracking more reliable, as overall the active marker devices will be visible more of the time. In fact, only a single active marker device needs to be temporally coded to identify all active marker devices in the marker cluster successfully.

Spatial and temporal coding can be combined to take advantage of the benefits of each method. In an embodiment, arranging one or more marker clusters on the object comprises arranging a plurality of marker clusters on the object, the plurality of temporally repeating patterns of first active marker devices in a first marker cluster is the same as the plurality of temporally repeating patterns of second active marker devices in a second marker cluster, and a spatial arrangement on the object of the first active marker devices is different from a spatial arrangement on the object of the second active marker devices. This further increases the number of distinguishable points that can be tracked, while requiring only a small number of spatially distinct marker clusters.

The method further comprises determining a spatial configuration of the object using the light detected by the one or more cameras. The spatial configuration may include a position of the object and/or an orientation of the object. Where the object is an articulated object (such as a human) comprising a number of segments that can move relative to one another, the spatial configuration of the object may comprise a position and/or orientation of all or a subset of the segments of the object.

In an embodiment, determining a spatial configuration of the object comprises a step S16 of determining an identity of at least one of the active marker devices based on the temporally repeating patterns of the active marker devices. In an embodiment where one or more marker clusters are used, determining a spatial configuration of the object may further comprise determining an identity of at least one of the marker clusters based on the pluralities of temporally repeating patterns of the one or more marker clusters. The identity of marker clusters may also be determined at least partially using the spatial arrangement of active marker devices within the marker cluster relative to other active marker devices within the same cluster. In an embodiment, algorithms for tracking rigid or articulated objects are used which rely on upfront knowledge of the marker cluster geometry only to help determine the correspondence between the markers on the object and the 3D reconstructions of the markers' positions.

Identifying the active marker devices or marker clusters is needed to initialise tracking of a particular object or segment of an object. In an embodiment, identification of the active marker device and/or marker clusters is performed initially at the beginning of a motion capture session. In an embodiment, identification is also performed at later times, for example if an active marker device or marker cluster is occluded during a motion capture session and tracking of its position and/or orientation is lost. In an embodiment, the identification is achieved by making use of two-dimensional (2D) or three-dimensional (3D) tracking, which gives continuity over time of the same physical active marker device or marker cluster, even after a time period (e.g. a time frame of the temporally repeating pattern) over which the active marker device was not emitting light.

In an embodiment, determining a spatial configuration of the object comprises a step S18 of determining a position and/or orientation of one or more active marker devices. The positions of active marker devices are determined using information including the position of the same active marker device within images captured at the same time by the one or more cameras, and information about the placement of the cameras, and their field of view. In an embodiment where marker clusters are used, determining a spatial configuration of the object may further comprise using information about the spatial relationship of active marker devices relative to other active marker devices within the same marker cluster. This is particularly useful in relation to determining the orientation of an active marker device or cluster. Where active marker devices within the same marker cluster have a relative spatial arrangement that is rotationally asymmetric, the arrangement of the marker cluster appearing in images captured by the cameras can be used to determine the position and orientation of the marker cluster.

Determining the position and/or orientation of active marker devices and/or marker clusters is performed continuously throughout the motion capture session. In an embodiment, identification of the active marker devices and/or marker clusters is also performed continuously. Although in FIG. 1 the steps of identifying the active marker devices and determining the position and/or orientation of the active marker devices are performed in series, these steps may be performed in parallel with each other and continuously throughout the motion capture session. This ensures that active marker devices that become occluded are rapidly re-identified when they become visible again, and also allows information about the positions of those active marker devices or marker clusters at previous times to inform the identification.

In an embodiment, determining a spatial configuration of the object further comprises using information about the arrangement of the active marker devices on the object. This can include a correspondence between active marker devices and segments of an articulated object. It may also include the position of the active marker device on the object, such that the extent of the object can be deduced from the position of the active marker device using information about the size and shape of the object. The orientation of an active marker device or marker cluster relative to the object or segment of an object can be also be used to determine the orientation and extent of the object.

In an embodiment, arranging one or more active marker devices on the object comprises arranging a plurality of active marker devices on the object, and using information about the arrangement of the active marker devices on the object comprises using constraints on the relative positions and/or orientations of the active marker devices, the constraints being based on the arrangement of the active marker devices on the object. For example, if two active marker devices are known to be arranged on the same segment of the object being tracked, then their determined positions must be consistent with the information about their placement on the segment and the size and shape of the segment. In addition, constraints regarding the allowable movement of joints connecting segments may be used to correct or inform the determined positions and/or orientations. These constraint can also be used in identifying active marker devices, as is discussed in further detail below. In an embodiment where the object is a human, animal, or robot, determining a spatial configuration of the object may comprise determining a pose of the human, animal, or robot. This may take into account constraints on joint movement and segment properties as mentioned above.

When tracking articulated objects (such as people or animals), the temporally repeating patterns of the active marker devices can be designed so that during every time frame, sufficient active marker devices are illuminated to be able to estimate the angles of the joints of the articulated model of the tracked object. i.e. every segment of the articulation is left with sufficient visible information to determine its position and orientation.

The method described above may be carried out using a motion tracking system such as that shown in FIG. 5. The motion tracking system 20 of FIG. 5 comprises one or more (in this case four) active marker devices 2 configured to be arranged on an object, the active marker devices 2 being further configured to emit light and each having an associated temporally repeating pattern comprising a plurality of time frames. The motion tracking system 20 further comprises a control system 26 configured to control the active marker devices 2 to emit light according to their respective temporally repeating patterns, wherein the temporally repeating patterns are such that the active marker devices 2 do not emit light during at least one time frame of the plurality of time frames. The motion tracking system 20 comprises one or more (in this case two) cameras 24 configured to detect light emitted by the active marker devices 2, and determining means 26 configured to determine a spatial configuration of the object using the light detected by the one or more cameras 24.

While objects can be identified successfully via temporal coding using temporally repeating patterns or geometric means using spatially distinguishable arrangements of active marker devices, as discussed above, there are scenarios in which it is advantageous to deploy external physical constraints to enable higher numbers of object to be used. Therefore, according to a second aspect of the invention, there is provided a method of motion tracking, an embodiment of which is shown in FIG. 6. The method of the second aspect may be used in combination with the first aspect described above, where temporal coding of active marker devices is used as part of the identification of the active marker devices. However, this is not essential, and the identification of active marker devices may be based entirely on spatial information about the active marker devices such as their position and/or orientation. The method comprises a step S10 of arranging a plurality of active marker devices on an object, the active marker devices being configured to emit light, and a step S14 of detecting light emitted by the plurality of active marker devices using one or more cameras. The active marker devices and the cameras are configured as described above. However, while using the method of the second aspect with temporally coded active markers may further increase the number of active marker devices that can be used simultaneously, it is not a requirement for the method of the second aspect that the active marker devices are configured to emit light according to a temporally repeating pattern.

The method of the second aspect further comprises determining a spatial configuration of the object using the light detected by the one or more cameras, wherein determining a spatial configuration of the object comprises using information about the arrangement of the active marker devices on the object to define constraints on the relative positions of the active marker devices, and identifying at least a subset of the plurality of active marker devices using the light detected by the one or more cameras and the constraints on the relative positions of the active markers. Constraints on the relative positions of active marker devices can be used when, for example, sets of active marker devices are attached to individual people, which put a limit on how far apart the active marker devices on that person can get from each other. Active marker devices are not identified using the determined constraints alone, but the determined constraints are combined with other sources of information. For example, temporally repeating patterns may be used according to the first aspect of the invention, or active marker devices may be arranged in clusters and their spatial arrangement used as part of identifying the active markers. The use of additional determined constraints allows spatial arrangements or temporally repeating patterns of active marker devices to be reused, because active marker devices will still be successfully identified due to the determined constraints and their proximity to other active marker devices that will uniquely identify a particular person.

For example, in an embodiment, the method further comprises a step S18 of determining a position and/or orientation of the plurality of active marker devices, and arranging a plurality of active marker devices on an object comprises arranging a first plurality of active marker devices on a first object and arranging a second plurality of active marker devices on a second object. Although in FIG. 6 the steps relating to identifying active marker devices and determining the position and/or orientation of the active marker devices are performed in series, these steps may be performed in parallel with each other and continuously throughout the motion capture session

In this case, identifying at least a subset of the plurality of active marker devices comprises determining whether an active marker device is part of the first plurality of active marker devices or part of the second plurality of active marker devices using the position and/or orientation of the active marker device and the constraints on the relative positions of the active marker devices. In this embodiment, at least some of the active marker devices are non-unique, but which of the objects they are on can still be identified using constraints on the relative positions of the active marker devices. In particular, this can be used where the plurality of active marker devices comprises a plurality of marker clusters, each comprising a plurality of active marker devices. In such a case, the spatial arrangements of the active marker devices within the marker clusters need not be unique for the marker clusters to be identifiable, as non-unique marker clusters can be identified using the constraints on their positions relative to other active marker devices. Either or both of the first and second pluralities of active marker devices may comprise a plurality of marker clusters.

As well as identifying active marker devices or marker clusters purely based on constraints on relative position, they may also be identified based on their positions relative to unique active marker devices or marker clusters. In an embodiment, the first plurality of active marker devices comprises a unique subset of one or more active marker devices, the unique subset being distinguishable from any other subset of the active marker devices. The unique subset may be a marker cluster as described above. In an embodiment, the unique subset comprises a plurality of active marker devices, and the active marker devices within the unique subset are arranged in a unique spatial configuration, the unique spatial configuration being distinguishable from the spatial configuration of any other subset of the active marker devices. Alternatively, the unique subset may be uniquely identifiable by comprising one or more temporally-coded active marker devices. In such an embodiment, identifying at least a subset of the plurality of active marker devices comprises a step S20 of identifying the unique subset, and determining whether an active marker device of the plurality of active marker devices is part of the first plurality of active marker devices using the position and/or orientation of the active marker device relative to the unique subset and the constraints on the position of the active marker device relative to the unique subset. In an embodiment, determining whether an active marker device is part of the first plurality of active marker devices comprises a step S22 of identifying one or more non-unique subsets of active marker devices, and a step S24 of determining whether a marker cluster comprising a plurality of active marker devices is part of the first plurality of active marker devices, i.e. which of the first and second pluralities the non-unique subset is a member of.

Identifying potentially non-unique marker clusters using their position relative to one or more unique subsets or unique marker clusters can be advantageous where several similar or identical objects need to be used within the motion capture session. For example, the object to be tracked may comprise a human holding a prop (such as an imitation weapon, item of luggage etc.). The plurality of active marker devices may be arranged so that active marker devices are placed on both the prop and the human. Using the above method, a number of such props can be used simultaneously and identified by their positions relative to unique subsets of active marker devices placed on the human. This would allow several people to interact with identical props in a scene, allowing the props to be uniquely identified without the need to provide unique arrangements of active marker devices on each instance of the same prop.

1. A method of motion tracking comprising:
arranging one or more active marker devices on an object, the active marker devices being configured to emit light and each having an associated temporally repeating pattern comprising a plurality of time frames;
controlling the one or more active marker devices to emit light according to their respective temporally repeating patterns, wherein the temporally repeating patterns are such that the active marker device does not emit light during at least one time frame of the plurality of time frames;
detecting light emitted by the one or more active marker devices using one or more cameras; and
determining a spatial configuration of the object using the light detected by the one or more cameras.

2. The method of claim 1, wherein the time frames of the temporally repeating patterns all have the same duration.

3. The method of claim 1, wherein the temporally repeating patterns are such that the active marker devices emit light during at least one time frame of every pair of consecutive time frames and/or (ii) for at least 25% of a duration of the temporally repeating patterns.

4. The method of any preceding claim 1, wherein:
the plurality of time frames of the temporally repeating patterns of at least a subset of the active marker devices includes tracking frames during which the active marker devices emit light;
the tracking frames of the temporally repeating patterns of all of the subset of active marker devices occur simultaneously; and
every nth time frame of the temporally repeating patterns of the subset of the active marker devices is a tracking frame, where n is an integer greater than or equal to 2.

5. The method of any preceding claim 1, wherein the active marker devices are controlled such that for each of the plurality of time frames of the temporally repeating patterns, the active marker devices either emit light for the entire duration of the time frame, or do not emit light for the entire duration of the time frame.

6. (canceled)

7. The method of claim 1, wherein the step of controlling the active marker devices comprises controlling the active marker devices such that the time frames of the temporally repeating patterns are synchronised with the capturing of images by the one or more cameras.

8. The method of claim 1, wherein:
the active marker devices comprise a receiver configured to receive wireless signals; and
controlling the active marker devices comprises transmitting a synchronisation signal to the active marker devices and controlling the active marker devices to emit light based on the synchronisation signal.

9. The method of claim 1, wherein:
arranging one or more active marker devices on the object comprises arranging a plurality of active marker devices on the object having an associated plurality of temporally repeating patterns; and
the temporally repeating pattern of a first active marker device is different from the temporally repeating pattern of at least one other active marker device.

10. The method of claim 9, wherein the temporally repeating patterns of the plurality of active marker devices are such that, at any point in time, at least 30% of the active marker devices emit light.

11. The method of claim 9 or 10, wherein all of the temporally repeating patterns in the plurality of temporally repeating patterns have the same duration.

12. The method of claim 1, wherein arranging one or more active marker devices on the object comprises arranging one or more marker clusters on the object, the marker clusters comprising a plurality of active marker devices having an associated plurality of temporally repeating patterns.

13. The method of claim 12, wherein each of the active marker devices within a marker cluster has a fixed spatial relationship with the other active marker devices within the same cluster.

14. The method of claim 13, wherein all of the active marker devices within a marker cluster are mounted on a single rigid surface.

15. The method of claim 12, wherein:
arranging one or more marker clusters on the object comprises arranging a plurality of marker clusters on the object; and
the plurality of temporally repeating patterns of active marker devices in a first cluster is different from the plurality of temporally repeating patterns of active marker devices in at least one other cluster.

16. The method of claim 12, wherein for at least one of the one or more marker clusters, the temporally repeating patterns of all the active marker devices in the marker cluster are the same.

17. The method of claim 12, wherein:
arranging one or more marker clusters on the object comprises arranging a plurality of marker clusters on the object;
the plurality of temporally repeating patterns of first active marker devices in a first marker cluster is the same as the plurality of temporally repeating patterns of second active marker devices in a second marker cluster; and
a spatial arrangement on the object of the first active marker devices is different from a spatial arrangement on the object of the second active marker devices.

18. The method of claim 1, wherein:
arranging one or more active marker devices on the object comprises arranging one or more marker clusters on the object, the marker clusters comprising at least one active marker device having an associated temporally repeating pattern, and at least one fixed marker device configured to emit light; and
the method further comprises controlling the fixed marker device to continuously emit light.

19. The method of claim 12, wherein determining a spatial configuration of the object further comprises one or both of: (i) determining an identity of at least one of the marker clusters based on the pluralities of temporally repeating patterns of the one or more marker clusters; and (ii) using information about the spatial relationship of active marker devices relative to other active marker devices within the same cluster.

20. (canceled)

21. The method of claim 1, wherein determining a spatial configuration of the object comprises one or more of: (i) determining an identity of at least one of the active marker devices based on the temporally repeating patterns of the active marker devices; (ii) determining a position of one or more active marker devices; and (iii) determining an orientation of one or more active marker devices.

22. (canceled)

23. The method of claim 21, wherein determining a spatial configuration of the object further comprises using information about the arrangement of the active marker devices on the object.

24. The method of claim 23, wherein:
arranging one or more active marker devices on the object comprises arranging a plurality of active marker devices on the object; and
using information about the arrangement of the active marker devices on the object comprises using constraints on the relative positions and/or orientations of the active marker devices, the constraints being based on the arrangement of the active marker devices on the object.

25. The method of claim 1, wherein the object is a human, animal, or robot, and determining a spatial configuration of the object comprises determining a pose of the human, animal, or robot.

26. A motion tracking system comprising:
one or more active marker devices configured to be arranged on an object, the active marker devices being further configured to emit light and each having an associated temporally repeating pattern comprising a plurality of time frames;
a control system configured to control the active marker devices to emit light according to their respective temporally repeating patterns, wherein the temporally repeating patterns are such that the active marker devices do not emit light during at least one time frame of the plurality of time frames; one or more cameras configured to detect light emitted by the active marker devices; and
determining means configured to determine a spatial configuration of the object using the light detected by the one or more cameras.

27. An active marker device configured for use in the motion tracking system of claim 26, the active marker device comprising:
a light emitting unit configured to emit light;
receiving means configured to receive wireless signals from the control system of the motion tracking system; and
control means configured to control the emission of light by the light emitting unit based on the wireless signals received by the receiving means.

28. The method of claim 1, wherein determining a spatial configuration of the object comprises:
using information about the arrangement of the active marker devices on the object to derive constraints on the relative positions of the active marker devices; and
identifying at least a subset of the plurality of active marker devices using the light detected by the one or more cameras and the constraints on the relative positions of the active markers.

29. A method of motion tracking comprising:
arranging a plurality of active marker devices on an object, the active marker devices being configured to emit light;
detecting light emitted by the plurality of active marker devices using one or more cameras; and
determining a spatial configuration of the object using the light detected by the one or more cameras, wherein determining a spatial configuration of the object comprises:
using information about the arrangement of the active marker devices on the object to define constraints on the relative positions of the active marker devices; and
identifying at least a subset of the plurality of active marker devices using the light detected by the one or more cameras and the constraints on the relative positions of the active markers.

30. The method of claim 28, further comprising determining a position and/or orientation of the plurality of active marker devices, and wherein:
arranging a plurality of active marker devices on an object comprises arranging a first plurality of active marker devices on a first object and arranging a second plurality of active marker devices on a second object; and
identifying at least a subset of the plurality of active marker devices comprises determining whether an active marker device is part of the first plurality of active marker devices or part of the second plurality of active marker devices using the position and/or orientation of the active marker device and the constraints on the relative positions of the active marker devices.

31. The method of claim 30, wherein the first plurality of active marker devices comprises a unique subset of one or more active marker devices, the unique subset being distinguishable from any other subset of the active marker devices.

32. The method of claim 31, wherein:
the unique subset comprises a plurality of active marker devices; and
the active marker devices within the unique subset are arranged in a unique spatial configuration, the unique spatial configuration being distinguishable from the spatial configuration of any other subset of the active marker devices.

33. The method of claim 31 or 32, wherein identifying at least a subset of the plurality of active marker devices comprises:
identifying the unique subset; and
determining whether an active marker device of the plurality of active marker devices is part of the first plurality of active marker devices using the position and/or orientation of the active marker device relative to the unique subset and the constraints on the position of the active marker device relative to the unique subset.
